(19) United States
(12) Patent Application Publication
(10) Pub. No.: US 2003/0148481 A1
(43) Pub. Date: Aug. 7, 2003

(54) Method for kinetic resolution of racemates of alcohols having one or several stereogenic centers

(75) Inventors: Fritz Theil, Berlin (DE); Helmut Sonnenschein, Berlin (DE)

Correspondence Address: GUDRUN E. HUCKETT, LONSSTR. 53, WUPPERTAL, 42289, DE

(73) Assignee: ASCA GmbH Angewandte Synthesechemie Adlershof, Berlin (DE)

(21) Appl. No.: 10/322,756

(22) Filed: Dec. 19, 2002

(30) Foreign Application Priority Data
Dec. 19, 2001 (DE) 101 64 269.5

(51) Int. Cl. C12P 7/02

(52) U.S. Cl. 435/155

(57) ABSTRACT

The present intention relates to a method for kinetic resolution of racemates of alcohols with one or several stereogenic centers. The invention is particularly suitable for producing pharmaceutical agents or plant protective agents. According to the invention, first racemic alcohols are converted with fluorinated acylation agents into racemic fluorinated carboxylic acid esters. Subsequently, by lipase-catalyzed reaction of the racemic fluorinated carboxylic acid ester with alcohols the fluorous phase marking of the faster reacting enantiomer is canceled and that of the slower reacting enatiomer is maintained. Subsequently, the enantiomers are extractively separated by distribution between organic phase and fluorous phase.

The present invention relates to a method for kinetic resolution of racemates of alcohols with one or several stereogenic centers. The invention is applicable in particular for manufacturing pharmaceutical agents or plant protective agents.

The lipase-catalyzed kinetic resolution of racemates of alcohols by enantioselective esterification or by enantioselective hydrolysis or alcoholysis of chiral carboxylic acid esters is a well-established method in organic synthesis (F. Theil, Chem. Rev. 1995, 95, 2203-2227; U. T. Bornscheuer, R. J. Kazlauskas, Hydrolases in Organic Synthesis, Wiley-VCH, Weinheim, 1999). The slower reacting enantiomer is retained as an alcohol or carboxylic acid ester while the faster reacting enantiomer is obtained as carboxylic acid ester or alcohol. Conventionally, alcohol and carboxylic acid ester are separated by chromatography (F. Theil et al., J. Org. Chem. 1994, 59, 388-393).

Simpler separations are obtained only when, for example, alcohol and ester differ with regard to their solubility properties to such an extent that an extractive separation between aqueous phase and organic phase is possible, a method which can be realized however only in exceptional situations (P. Stead et al., Tetrahedron: Asymmetry 1996, 7, 2247-2250).

A further possibility for the separation of ester and alcohol is provided when the formed ester is acidic, which can be realized, for example, by esterification with cyclic carboxylic acid anhydrides. However, this is also not a generally applicable method because cyclic anhydrides do not constitute optimal acyl donors for lipase-catalyzed esterification reactions. Moreover, acidic compounds reduce the lipase activity (B. Berger et al. Tetrahedron: Asymmetry 1990, 1, 541-546; U. T. Bornscheuer, R. J. Kazlauskas, Hydrolases in Organic Synthesis, Wiley-VCH, pp. 44-47, Weinheim, 1999).

Moreover, a method for lipase-catalyzed resolution of racemates has been suggested where racemic alcohols are reacted with fluorinated acylation agents or fluorinated carboxylic acid esters of racemic alcohols are reacted with water, wherein a fluorous phase marking of the faster or slower reacting enantiomer is achieved and the enantiomers are subsequently extractively separated by means of distribution between the organic phase and the fluorous phase (F. Theil, H. Sonnenschein, PCT/DEO0/004536). However, hydrolyses can have disadvantages for certain substrates in comparison to reactions in an organic medium with respect to stability of the substrate and/or product in the aqueous medium, control of the reaction, isolation of the products, and recovery of the lipases. Methods for acylation of racemic alcohols circumvent these difficulties; however, it is known that the acylation of alcohols, the hydrolysis and the alcoholysis of carboxylic acid esters can occur with different selectivity, respectively (M. Ranchoux et al., Tetrahedron: Asymmetry: 1998, 9 ,581-587; K. Takabe et al., Tetrahedron Lett. 2000, 41, 9859-9863), and, accordingly, alcoholyses for certain substrates are not only advantageous in comparison to hydrolyses but also relative to acylation reactions.

It is therefore an object of the invention to provide an improved method for resolution of alcohols having one or several stereogenic centers.

According to the invention, this object is solved by a method for kinetic resolution of racemates of alcohols having one or several stereogenic centers according to which method racemic alcohols are first converted with fluorinated acylation agents into racemic fluorinated carboxylic acid esters. Subsequently, by lipase-catalyzed reaction of the racemic fluorinated carboxylic acid ester with alcohols, the fluorous phase marking of the faster reacting enantiomer is canceled and that of the slower reacting enantiomer is maintained. Subsequently, the enantiomers are extractively separated by distribution between organic phase and fluorous phase.

According to the invention, racemic alcohols with one or several stereogenic centers are converted with per-fluorinated acylation agents into an ester of the formula I

R—(CH₂)ₙ—COOCHR¹R² (I)

wherein

R is a per-fluorinated alkyl group such as —(CF₂)ₘ—CF₃

wherein m can be an integer from 3 to 18 or

a per-fluorinated aromatic group such as C₆F₄X and

X is fluorine or a per-fluorinated alkyl group

R¹, R² are alkyl, alkenyl, aryl, or heteroaryl and

n can be an integer from 0 to 4,

and, subsequently, as is known in the art, subjected with ester-cleaving enzymes, preferably lipases, to an enantioselective alcoholysis by means of an alcohol of the formula II

R³—OH (I)

wherein

R³ is an aliphatic alkyl group of one to twelve carbon atoms, a cycloaliphatic alkyl group of four to eight carbon atoms, or a benzyl group or aryl-substituted benzyl group.

According to the invention, as shown in the Schematic 1,

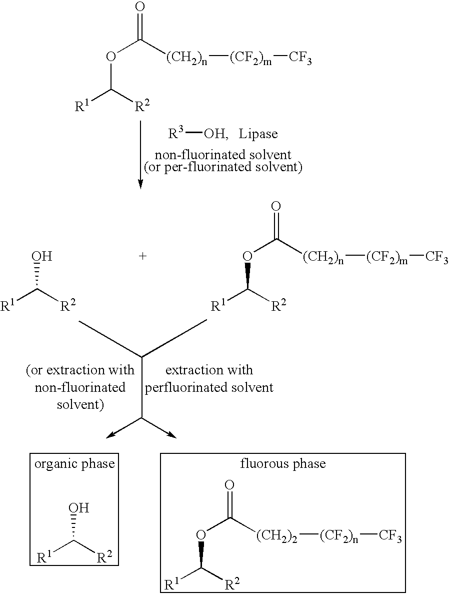

Schematic 1: R¹, R² and R³ as defined above

in the presence of ester-cleaving enzyme, preferably a lipase, the per-fluorinated group is cleaved off the faster reacting enantiomer, thereby making this enantiomer no longer soluble in fluorous solvents. During the subsequent distribution of the reaction products between organic phase and fluorous phase, the free alcohol is in the organic phase and the uncleaved ester in the fluorous phase in accordance with the invention.

According to the invention, the alcoholysis is carried out with a lipase, which is microbe-derived, plant-derived or animal-derived, either in a solvent conventional for this reaction, such as aliphatic or aromatic hydrocarbons, ethers, tertiary alcohols or also chlorohydrocarbons. Subsequently, the per-fluorinated enantiomer is extracted with a per-fluorinated solvent which is immiscible with the non-fluorinated organic solvent. As an alternative, the lipase-catalyzed reactions are carried out in a per-fluorinated solvent and, subsequently, the non-fluorinated enatiomer is extracted by means of a non-fluorinated organic solvent.

According to the invention, the lipase-catalyzed kinetic resolution of racemates can also be performed in a two-phase system of organic and fluorous solvents which are immiscible at room temperature or lower temperature. The phase homogenization during the chemical reaction is realized by heating. The phase separation, and thus the product separation correlated therewith, is achieved by cooling the reaction mixture to a temperature below the phase mixing temperature.

In this way, the separation of enantiomers can be performed in good yields.

By means of the following examples, the invention will be explained in more detail.

### EXAMPLE 1

To a solution of racemic 4,4,5,5,6,6,7,7,8,8,9,9,10,10,11,11,11-heptadecafluoro undecanoic acid-1-phenylethylester (1.79 g, 3 mmol) and n-butanol (0.89 g, 12 mmol) in acetonitrile (70 ml), Candida antarctica B lipase (5 g) is added and the solution is stirred until 50% of the ester has reacted. The enzyme is filtered off, and the filtrate is concentrated under vacuum until dry. The residue is dissolved in methanol (10 ml) and the solution is extracted six times with n-perfluorohexane. The organic phase contains (R)-1-phenyl ethanol with an enantiomer excess of >95%. The fluorous phase contains (S)-4,4,5,5,6,6,7,7,8,8,9,9,10,10,11,11,11-heptadecafluoro undecanoic acid-1 -phenylethylester with an enantiomeric excess of >95%.

### EXAMPLE 2

Candida antarctica B lipase (5 g) is added to a solution of racemic 4,4,5,5,6,6,7,7,8,8,9,9,10,10,11,11,11-heptadeca fluoro undecanoic acid-indan-1-yl ester (1.82 g, 3 mmol) and n-butanol (0.89 g, 12 mmol) in acetonitrile (70 ml), and the solution is stirred until 50% of the ester has reacted. The enzyme is filtered off, and the filtrate is concentrated under vacuum until dry. The residue is dissolved in methanol (10 ml) and the solution is extracted six times with n-perfluorohexane. The organic phase contains (R)-1-indanol with an enantiomer excess of >95%. The fluorous phase contains (S)-4,4,5,5,6,6,7,7,8,8,9,9,10,10,11,11,11-heptadecafluoro undecanoic acid indan-1-yl ester with an enantiomer excess of ≧90%.

### EXAMPLE 3

Candida antarctica B lipase (5 g) is added to a solution of racemic 4,4,5,5,6,6,7,7,8,8,9,9,10,10,11,11,11 -heptadecafluoro undecanoic acid 1 -methyl-3-trimethylsilanyl-prop-2-inyl ester (1.85 g, 3 mmol) in acetonitrile (70 ml) and n-butanol (0.89 g, 12 mmol), and the solution stirred until 40% of the ester has reacted. The enzyme is filtered off, and the filtrate is concentrated under vacuum until dry. The residue is dissolved in methanol (10 ml) and the solution is extracted six times with n-perfluorohexane. The organic phase contains (R)-4-trimethlylsilanyl-but-3-in-2-ol with an enatiomer excess of >99%. The fluorous phase contains (S)-4,4,5,5,6,6,7,7,8,8,9,9,10,10,11,11,11-heptadecafluoro undecanoic acid 1-methyl-3-trimethylsilanyl-prop-2-inyl ester with an enantiomer excess of 68%.

1. A method for kinetic resolution of racemates of alcohols having one or several stereogenic centers, wherein racemic alcohols are first converted with fluorinated acylation agents into racemic fluorinated carboxylic acid esters, characterized in that
by lipase-catalyzed reaction of the racemic fluorinated carboxylic acid ester with alcohols the fluorous phase marking of the faster reacting enatiomer is canceled and that of the slower reacting enantiomer is maintained and that, subsequently, the enantiomers are extractively separated by distribution between organic phase and fluorous phase.

2. The method according to claim 1, characterized in that for kinetic resolution of racemates of alcohols having one or several stereogenic centers racemic alcohols are converted with fluorinated acylation agents into their esters of the formula I
R—(CH₂)ₙ—COOCHR¹R² (I)
wherein
R is a per-fluorinated alkyl group such as —(CF₂)ₘ—CF₃
wherein m can be an integer from 3 to 18 or
a per-fluorinated aromatic group such as C₆F₄X and
X is fluorine or a per-fluorinated alkyl group,
R¹, R² are alkyl, alkenyl, aryl, or heteroaryl and
n can be an integer from 0 to 4,
subsequently, these esters are subjected to an enantioselective alcoholysis with an alcohol of the formula II
R³—OH (II)
wherein
R³ is an aliphatic alkyl group of one to twelve carbon atoms, a cycloaliphatic alkyl group of four to eight carbon atoms, or a benzyl group or aryl-substituted benzyl group,
and the obtained enantiomers are separated from one another by extractive distribution between organic phase and fluorous phase.

3. The method according to claim 1, characterized in that the lipase is microbe-derived, plant-derived, or animal-derived.

4. The method according to claim 2, characterized in that the alcoholysis is carried out in aliphatic or aromatic hydrocarbons, ethers, tertiary alcohols, or chlorohydrocarbons and the extraction of the fluorinated enantiomers is carried out with a per-fluorinated solvent.

5. The method according to claim 2, characterized in that the alcoholysis is carried out in a perfluorinated solvent and the extraction is carried out in a non-fluorinated solvent.

6. The method according to claim 2, characterized in that the lipase-catalyzed alcoholysis is carried out in a two-phase system of organic phase and fluorous phase, wherein the phase homogenization during the reaction is achieved by heating and, subsequently, the phase separation and thus the product separation is carried out by cooling the reaction mixture to a temperature below the phase mixing temperature.

7. The method according to claim 4, characterized in that the solvents employed in the lipase-catalyzed alcoholysis are distilled off after stopping the reaction and subsequently a distribution of the products between organic phase and fluorous phase takes place.

8. The method according to claim 3, characterized in that the alcoholysis is carried out in aliphatic or aromatic hydrocarbons, ethers, tertiary alcohols, or chlorohydrocarbons and the extraction of the fluorinated enatiomer is carried out with a per-fluorinated solvent.

9. The method according to claim 3, characterized in that the alcoholysis is carried out in a per-fluorinated solvent and the extraction is carried out with a non-fluorinated solvent.

10. The method according to claim 3, characterized in that the lipase-catalyzed alcoholysis is carried out in a two-phase system of organic phase and fluorous phase, wherein the phase homogenization during the reaction is achieved by heating and, subsequently, the phase separation and thus the product separation is carried out by cooling the reaction mixture to a temperature below the phase mixing temperature.
